(19) United States
(12) Patent Application Publication
(10) Pub. No.: US 2006/0155290 A1
(43) Pub. Date: Jul. 13, 2006

(54) Instrument for reconstructing ligament and method of reconstructing ligament

(76) Inventor: Konsei Shino, Suita-shi (JP)

Correspondence Address: NIXON & VANDERHYE, PC, 901 NORTH GLEBE ROAD, 11TH FLOOR, ARLINGTON, VA, 22203, US

(21) Appl. No.: 10/563,915

(22) PCT Filed: Jul. 8, 2004
(86) PCT No.: PCT/JP04/09741

§ 371 Date: Jan. 10, 2006

(30) Foreign Application Priority Data
Jul. 11, 2003 (JP) 2003196055

(51) Int. Cl. A61F 5/00

(52) U.S. Cl. 606/86

(57) ABSTRACT

A dilator (1) for reconstructing an anterior cruciate ligament, comprising a tip part (4) having two through-holes (2) and (3) arranged in juxtaposition and parallel with each other, a body part (5) and a rear end part (8) having two through-holes (6) and (7) arranged in juxtaposition and coaxially with the two through-holes (2) and (3), respectively. The tip part (4) and the body part (5) are formed in uniform generally elongated shapes in cross section longer in the arranged direction of the through-holes (2) and (3) so that a flat tunnel can be formed by striking the instrument from the rear end part (8) thereof to drive the tip part (4) and the body part (5) into an articular bone part. Thus, the anterior cruciate ligament can be reconstructed to a more tough ligament similar to a normal one in a shorter time.

### TECHNICAL FIELD

The present invention relates to a ligament reconstruction device and a ligament reconstruction method. More specifically, the invention relates to a ligament reconstruction device and a ligament reconstruction method to be utilized for forming a ligament fixation tunnel in an articular bone when a damaged ligament such as an anterior cruciate ligament (a ligament connecting a femur and a tibia) is reconstructed.

### BACKGROUND ART

In conventional anterior cruciate ligament reconstruction (technique), for example, a bone tunnel is formed in a tibia as extending through a bone wall of the tibia, and a cylindrical bone tunnel having a round cross section is formed in a femur as extending through a bone wall of the femur. A looped ligament graft is inserted through these bone tunnels with one end portion thereof fixated to the femur via a metal fixture. A body of a hollow cylindrical metal screw is inserted into the bone tunnel of the tibia from an open end of the bone tunnel (see Japanese Unexamined Patent Publication No. 2001-25478).

The anterior cruciate ligament reconstruction technique has been steadily developed. This technique is applied to an increased number of reconstruction operations, and makes it possible to properly perform the operations with a reduced rate of occurrence of complications. Therefore, the anterior cruciate ligament reconstruction is now a standard treatment with an excellent treatment effect.

Young and healthy athletes often need the anterior cruciate ligament reconstruction. Therefore, it is constantly demanded to reconstruct a more robust ligament in a shorter period of time.

### DISCLOSURE OF THE INVENTION

As a result of intensive studies in view of the foregoing, the inventor of the present invention has found that there is a great gap between a ligament graft having a generally rectangular or elliptical cross section (e.g., having a minor axis of 4 mm and a major axis of 8 mm) rather than a round cross section and an interior surface of a conventionally formed round bone tunnel (e.g., having an inner diameter of 8 to 10 mmφ), and reduction of the gap facilitates the bonding of the ligament graft to a bone. Thus, the present invention has been attained.

According to the present invention, there is provided a ligament reconstruction device, which comprises a tip and body portion having two parallel through-holes formed therein in juxtaposition, and a rear end portion having two through-holes formed therein in juxtaposition coaxially with the two through-holes of the tip and body portion. The tip and body portion has a uniform and generally elliptical or rectangular cross section elongated in a direction in which the through-holes thereof are juxtaposed, so that the tip and body portion is driven into an articular bone to form a flat tunnel in the bone by hitting the rear end portion.

According to the present invention, the tip and body portion has a generally elliptical or rectangular cross section rather than a simple round cross section. Therefore, the bone cavity to be formed in the bone has a cross section close to the cross section of an ordinary ligament, so that the outer peripheral surface of the ligament is entirely brought into proximity to the interior surface of the bone cavity. Hence, the ligament can be bonded to the bone with a greater bonding force in a shorter period of time.

According to another aspect of the present invention, there is provided a ligament reconstruction method utilizing the aforesaid ligament reconstruction device, the method comprising the steps of: drilling a center guide pin into an articular bone and over-drilling the guide pin to a predetermined depth; drilling two guide pins into the bone parallel to the center guide pin and then removing the center guide pin; over-drilling the two guide pins; and driving the tip and body portion of the ligament reconstruction device into the articular bone toward an insertion site of the ligament graft of the articular bone by hitting the rear end portion of the ligament reconstruction device to form a flat tunnel into which one end portion of a ligament is to be inserted.

### BRIEF DESCRIPTION OF THE DRAWINGS

FIG. 1 is a front view illustrating a ligament reconstruction device according to one embodiment of the present invention;

FIG. 2 is a sectional view taken along a line A-A in FIG. 1;

FIG. 3 is a right side view of the ligament reconstruction device of FIG. 1;

FIG. 4 is an explanatory diagram illustrating a ligament reconstruction method utilizing the ligament reconstruction device shown in FIGS. 1 to 3;

FIG. 5 is an explanatory diagram illustrating the next step of the ligament reconstruction method utilizing the ligament reconstruction device;

FIG. 6 is an explanatory diagram illustrating the next step of the ligament reconstruction method utilizing the ligament reconstruction device;

FIG. 7 is an explanatory diagram illustrating the next step of the ligament reconstruction method utilizing the ligament reconstruction device;

FIG. 8 is an explanatory diagram illustrating the next step of the ligament reconstruction method utilizing the ligament reconstruction device;

FIG. 9 is an explanatory diagram illustrating the next step of the ligament reconstruction method utilizing the ligament reconstruction device;

FIG. 10 is an explanatory diagram illustrating the next step of the ligament reconstruction method utilizing the ligament reconstruction device;

FIG. 11 is an explanatory diagram illustrating the next step of the ligament reconstruction method utilizing the ligament reconstruction device;

FIG. 12 is an explanatory diagram illustrating another ligament reconstruction method utilizing the ligament reconstruction device;

FIG. 13 is an explanatory diagram illustrating the next step of the another ligament reconstruction method utilizing the ligament reconstruction device; and

FIG. 14 is an explanatory diagram illustrating the next step of the another ligament reconstruction method utilizing the ligament reconstruction device.

### BEST MODE FOR IMPLEMENTING THE INVENTION

A ligament reconstruction device according to the present invention mainly includes a tip and body portion, and a rear end portion.

The tip and body portion has two parallel through-holes formed therein in juxtaposition, and has a generally elliptical or rectangular cross section elongated in a direction in which the two through-holes are juxtaposed.

The generally elliptical or rectangular cross section preferably has a major axis/minor axis ratio of 2 to 5. The elliptical cross section is preferably of a generally oval shape or a racetrack-like elliptical shape. The racetrack-like elliptical shape is preferably defined by a pair of parallel straight lines spaced a distance of 3 to 6 mm from each other and each having a length of 4 to 8 mm and a pair of semicircles connecting opposite ends of these straight lines. The rectangular cross section preferably has a minor edge length of 3 to 6 mm and a major edge length of 7 to 14 mm. Therefore, the tip and body portion preferably has a cross sectional area of 21 to 84 mm².

The tip and body portion preferably has a length of 100 to 200 mm.

The rear end portion has two through-holes which are respectively coaxial with the two through-holes of the tip and body portion, and is typically of a thick elliptical or round disk shape having a greater cross sectional area than the tip portion. The rear end portion is hit by a hummer to drive the tip portion into the articular bone. Thus, a flat tunnel having a cross section conformable to the cross section of the tip and body portion is formed in the articular bone.

Next, an exemplary method for ligament reconstruction to be performed with the use of the ligament reconstruction device having the aforesaid construction will be described.

(1) With the use of an ordinary drill guide, a center guide pin is drilled into the center of a ligament attachment portion of an articular bone.

(2) With the use of a drill having a diameter of 10 mmφ which is equal to the maximum transverse diameter of a dilator, the portion of the articular bone around the guide pin is over-drilled to a predetermined depth [to form a round hole].

(3) With the use of an ordinary offset pin guide, two guide pins are drilled in parallel relation into the bone on opposite sides of the center guide pin.

(4) The center guide pin is removed.

(5) Portions of the bone around the two guide pins are removed by over-drilling.

(6) The rear end portion of the inventive ligament reconstruction device is hit to drive the tip and body portion of the ligament reconstruction device into the articular bone toward a cortex of the articular bone on an opposite side. Thus, a flat tunnel into which one end portion of a ligament is to be inserted is formed in an innermost portion of the round hole.

The round hole desirably has a depth of 10 to 30 mm, and the flat tunnel desirably has a depth of 10 to 30 mm. Where the ligament reconstruction is reconstruction of an anterior cruciate ligament, the articular bone is a tibia. An artificial ligament may be used as the ligament, but a ligament graft, particularly a patellar tendon of a human (a patient or a dead person) with bone plugs, is preferred.

An embodiment of the present invention will hereinafter be described in detail with reference to the attached drawings.

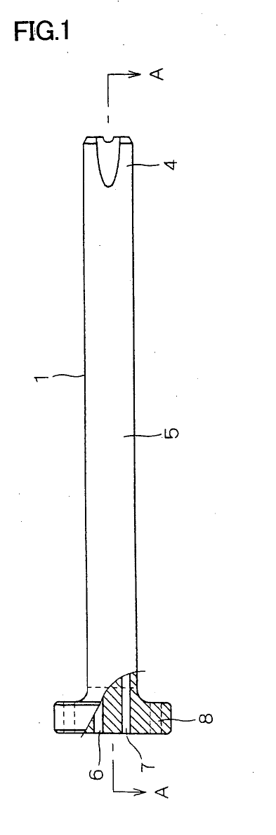

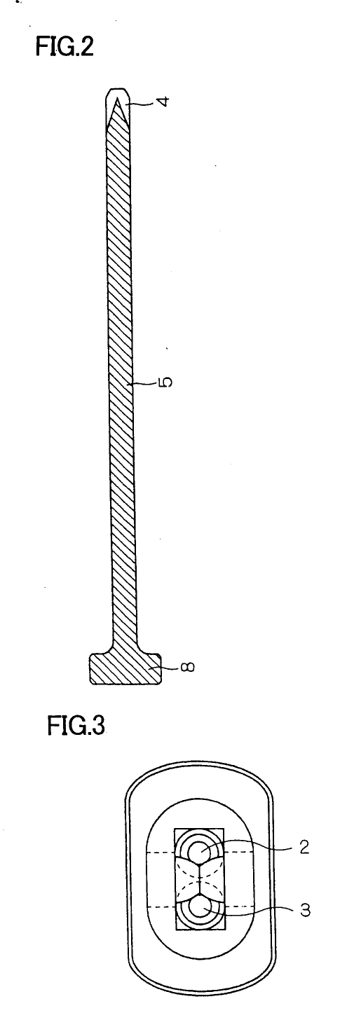

FIG. 1 is a front view illustrating a ligament reconstruction device according to one embodiment of the present invention. FIG. 2 is a sectional view taken along a line A-A in FIG. 1, and FIG. 3 is a side view of the ligament reconstruction device of FIG. 1.

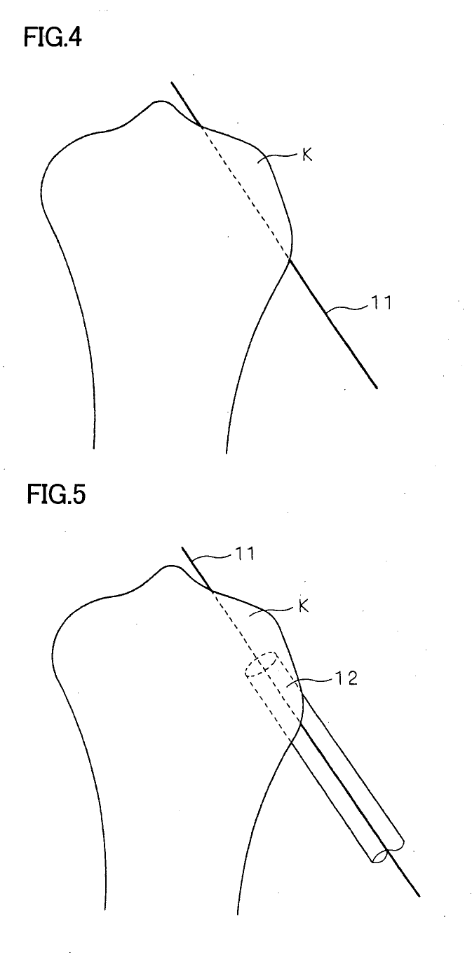

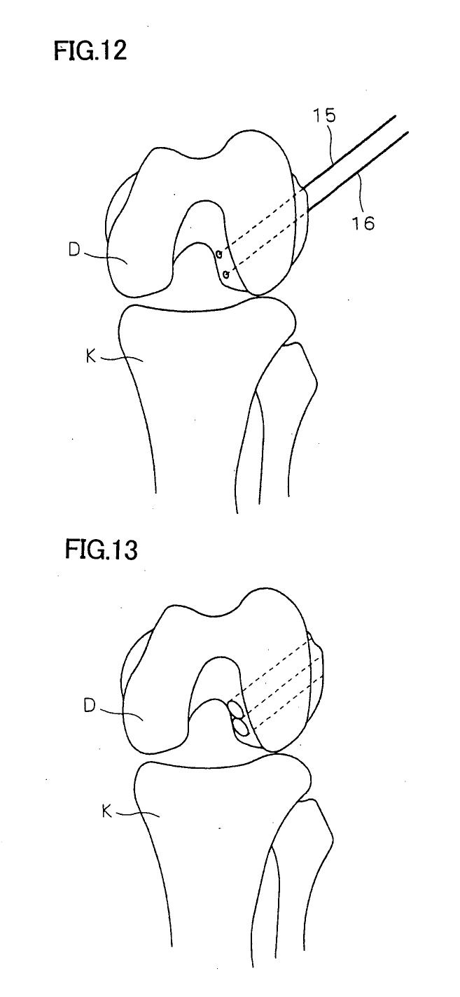

FIGS. 4 to 11 are explanatory diagrams illustrating a ligament reconstruction method utilizing the inventive ligament reconstruction device. FIGS. 12 to 14 are explanatory diagrams illustrating another ligament reconstruction method utilizing the inventive ligament reconstruction device.

Referring first to FIGS. 1 to 3, an anterior cruciate ligament reconstruction dilator 1 as the inventive ligament reconstruction device includes a tip and body portion 4, 5 having two parallel through-holes 2, 3 formed therein in juxtaposition, and a rear end portion 8 having two through-holes 6, 7 formed therein coaxially with the two through-holes 2, 3.

The tip and body portion 4, 5 has an elliptical cross section which is elongated in a direction in which the through-holes 2, 3 are juxtaposed. More specifically, the tip and body portion 4, 5 has a generally racetrack-shaped elliptical cross section (like an athletic track). The rear end portion 8 has a generally racetrack-shaped elliptical cross section which is larger than the cross section of the tip and body portion 4, 5.

The through-holes 2, 3, 6, 7 each have an inner diameter of 2.5 mmφ. Straight lines partly defining the cross section of the tip and body portion 4, 5 are spaced a distance of 5 mm from each other, and each have a length of 6 mm. Straight lines partly defining the cross section of the rear end portion 8 are spaced a distance of 15 mm from each other, and each have a length of 17 mm.

A tip portion 4 of the tip and body portion 4, 5 has front and rear cutaway portions 9, 10, so that the tip and body portion 4, 5 can be smoothly driven into the articular bone from a cortex of the articular bone toward a ligament attachment portion inside a joint by hitting the rear end portion 8 of the ligament reconstruction dilator 1.

An anterior cruciate ligament reconstruction method to be performed with the use of the anterior cruciate ligament reconstruction dilator 1 having the aforesaid construction will next be described with reference to FIGS. 4 to 11.

(1) With the use of an ordinary drill guide, a single 2.4-mmφ guide pin 11 (a center guide pin in FIG. 4) is drilled into a tibia K from an anterior medial cortex of the tibia K toward the center of a portion of the tibia K to which an anterior cruciate ligament is to be attached.

(2) With the use of a 10-mmφ drill, the tibia K is over-drilled to a depth of 20 mm along the guide pin 11 from an anterior surface of the tibia K to form a round hole 12 (see FIG. 5).

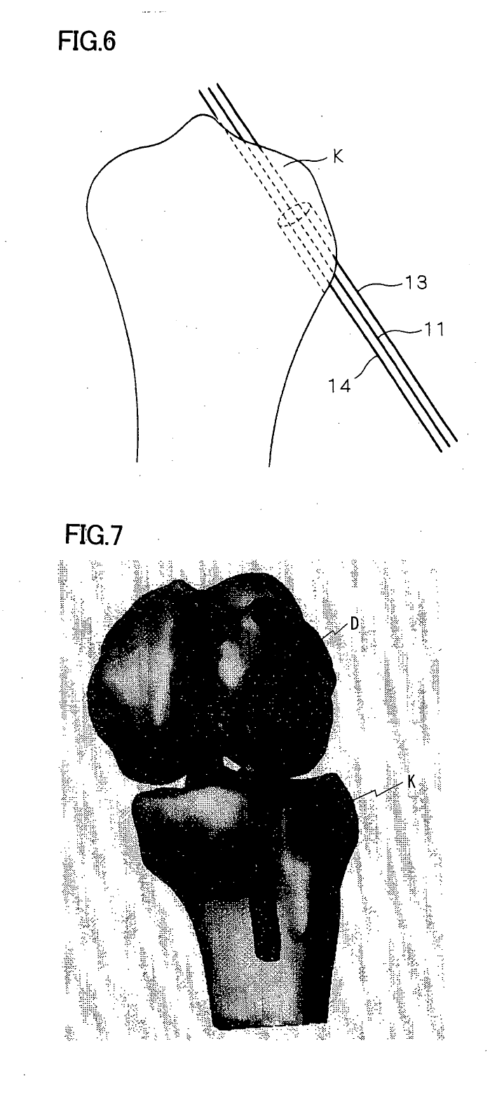

(3) With the use of an ordinary offset pin guide, two guide pins 13, 14 are drilled in parallel relation into the tibia K at positions which are 3 mm anteromedially and posterolaterally apart from the center guide pin 11 (see FIGS. 6 and 7). A reference character D denotes a femur.

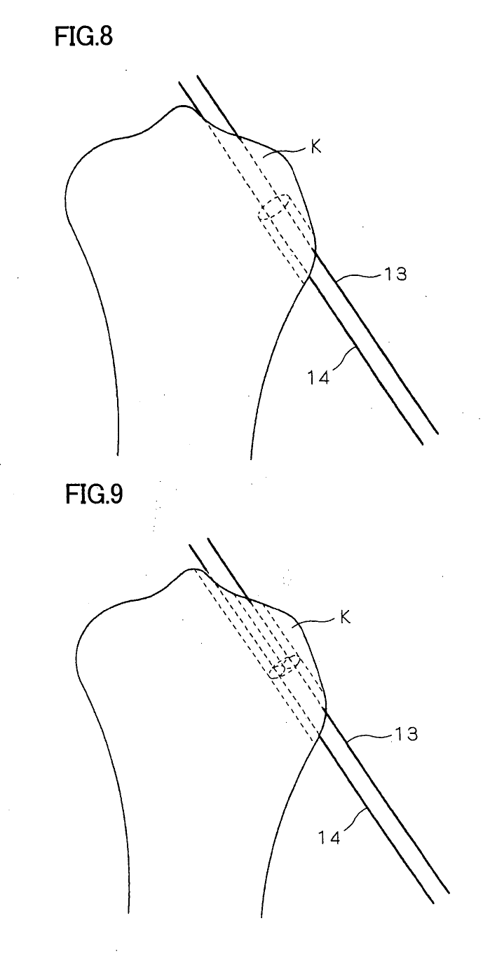

(4) The center guide pin 11 is removed (see FIG. 8).

(5) With the use of a 5 mm cannulated drill, the tibia is over-drilled along the two guide pins 13, 14 to an articular surface (see FIG. 9).

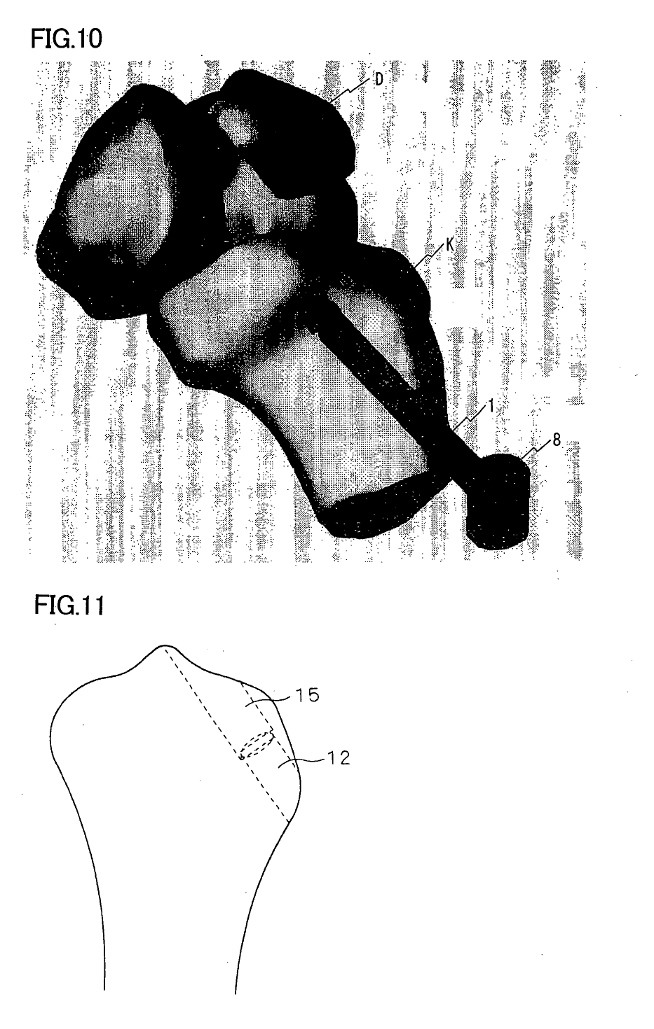

(6) With the two guide pins 13, 14 fitted in the through-holes 2, 3, 6, 7 of the anterior cruciate ligament reconstruction dilator 1, the rear end portion 8 of the anterior cruciate ligament reconstruction dilator 1 is hit by a hummer to drive the tip and body portion 4, 5 into the tibia K toward a ligament attachment portion of the tibia K. Thus, a flat tunnel 15 having a size of about 5 mm×about 10 mm is formed (see FIGS. 10 and 11).

Another anterior cruciate ligament reconstruction method to be performed with the use of the anterior cruciate ligament reconstruction dilator 1 will next be described with reference to FIGS. 12 to 14.

(1) With the use of an ordinary drill guide, a single 2.4-mmφ guide pin 16 is drilled into a femur D from a lateral cortex of the femur D toward the center of a portion of the femur D (a 9 o'clock position of a right knee, a 9 o'clock position of a left knee or a position 6 mm apart from a posterior margin of an intercondylar notch) to which posterolateral fibers of an anterior cruciate ligament are bonded. A reference character K denotes a tibia.

(2) A 20-mm long incision centering on the guide pin 16 is made in a skin on a lateral side of the femur D.

(3) With the use of an ordinary offset parallel pin guide, a second guide pin 17 is drilled into the femur D as being spaced 6 mm from the first guide pin in parallel relation in a noon direction (see FIG. 12).

(4) With the use of a 5 mm cannulated drill, the femur is over-drilled along the two guide pins 16, 17 from a lateral cortex to an articular surface (see FIG. 13).

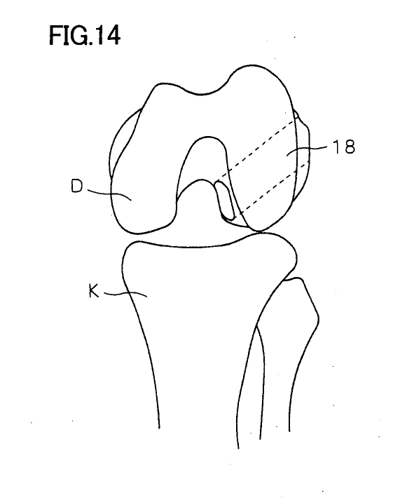

(5) The rear end portion 8 of the anterior cruciate ligament reconstruction dilator 1 is hit by a hummer to drive the tip and body portion 4, 5 into the femur D toward a medial cortex of the lateral chondyle of the lateral chondyle of the femur D. Thus, a flat tunnel 18 having a size of about 5 mm×about 10 mm is formed (see FIG. 14).

One end portion of a patellar tendon with a bone piece is inserted into the flat tunnel 15 formed in the tibia K by the former ligament reconstruction method via the round hole 12 by the conventional method, and fixated in the flat tunnel 15 by a button or a screw. The other end portion of the patellar tendon with the bone piece is properly introduced into the flat tunnel 18 formed in the femur D by the latter ligament reconstruction method through the round hole 12 and the flat tunnel 15, and fixated in the flat tunnel 18 by a button or a screw.

According to the present invention, as described above, the tip portion of the anterior cruciate ligament reconstruction device has a generally elliptical or rectangular cross section rather than a round cross section. Therefore, a bone cavity to be formed in a bone has a cross section close to the cross section of an ordinary ligament, so that the outer peripheral surface of the ligament is entirely brought into proximity to the interior surface of the bone cavity. Hence, the ligament can be bonded to the bone with a greater contact area in a shorter period of time.

1. A device for ligament reconstruction comprising:
a tip and body portion having two parallel through-holes formed therein in juxtaposition; and
a rear end portion having two through-holes formed therein in juxtaposition coaxially with the two through-holes of the tip and body portion,
wherein the tip and body portion has a uniform and generally elliptical or rectangular cross section elongated in a direction in which the through-holes thereof are juxtaposed, so that the tip and body portion is driven into an articular bone to form a flat tunnel in the bone by hitting the rear end portion.

2. A ligament reconstruction device as set forth in claim 1, wherein the generally elliptical or rectangular cross section has a major axis/minor axis ratio of 2 to 5.

3. A ligament reconstruction device as set forth in claim 1, wherein the elliptical cross section is of a generally oval shape or a racetrack-like elliptical shape.

4. A ligament reconstruction device as set forth in claim 3, wherein the racetrack-like elliptical shape is defined by a pair of parallel straight lines spaced a distance of 3 to 6 mm from each other and each having a length of 4 to 8 mm and a pair of semicircles connecting opposite ends of the straight lines.

5. A ligament reconstruction device as set forth in claim 1, wherein the rectangular cross section has a minor edge length of 3 to 6 mm and a major edge length of 7 to 14 mm.

6. A ligament reconstruction device as set forth in claim 1, wherein the tip and body portion has a cross sectional area of 21 to 84 mm².

7. A ligament reconstruction device as set forth in claim 1, wherein the ligament reconstruction is reconstruction of an anterior cruciate ligament.

8. A method for ligament reconstruction utilizing a ligament reconstruction device as recited in claim 1, the method comprising the steps of:
drilling a single center guide pin into an articular bone and over-drilling the guide pin to a predetermined depth;
drilling two guide pins into the bone parallel to the center guide pin and then removing the center guide pin;
over-drilling the two guide pins; and
driving the tip and body portion of the ligament reconstruction device into the articular bone from a cortex of the articular bone toward a ligament attachment portion inside a joint by hitting the rear end portion of the ligament reconstruction device to form a flat tunnel into which one end portion of a ligament is to be inserted.

9. A ligament reconstruction method as set forth in claim 8, wherein the ligament is an anterior cruciate ligament with a bone piece.

10. A ligament reconstruction method as set forth in claim 8, wherein the ligament reconstruction is reconstruction of an anterior cruciate ligament, and the articular bone is a tibia.
